(19) United States
(12) Patent Application Publication
(10) Pub. No.: US 2014/0373833 A1
(43) Pub. Date: Dec. 25, 2014

(54) ELECTRONIC CIGARETTE INHALATION ROD AND ELECTRONIC CIGARETTE USING THE SAME

(71) Applicant: KIMREE HI-TECH INC., Tortola (VG)

(72) Inventor: QIUMING LIU, Huizhou City (CN)

(21) Appl. No.: 14/480,789

(22) Filed: Sep. 9, 2014

Related U.S. Application Data
(63) Continuation-in-part of application No. PCT/CN2013/077312, filed on Jun. 17, 2013.

(51) Int. Cl. A24F 47/00; A61M 15/06

(52) U.S. Cl. CPC A24F 47/008; A61M 15/06; A61M 2205/587; A61M 2205/121; A61M 2205/123; A61M 2202/0007; A61M 2202/049
USPC 128/202.21

(57) ABSTRACT

The present invention relates to an electronic cigarette inhalation rod and an electronic cigarette. The electronic cigarette inhalation rod includes an inhalation tube and a first connector disposed at one end of the inhalation tube. The inhalation tube and the first connector are an integrally formed structure. Therefore, by the integral formed structure of the inhalation tube and the first connector, on one hand the first connector will not fall down from the inhalation rod, the product is of stable structure, good quality, and easy to use; on the other hand, the assembly is convenient and the appearance is beautiful. The electronic cigarette includes the inhalation rod and a battery rod. The electronic cigarette inhalation rod and the battery rod are matchingly connected.

### CROSS REFERENCE TO RELATED APPLICATIONS

This application is a U.S. continuation-in-part application under 35 U.S.C. § 111(a) claiming priority under 35 U.S.C. § § 120 and 365(c) to International Application No. PCT/CN2013/077312 filed Jun. 17, 2013, the contents of which are incorporated by reference herein in the entirety for all intended purposes.

### FIELD OF THE INVENTION

The present invention relates to an electronic cigarette inhalation rod and electronic cigarettes using the same.

### BACKGROUND OF THE INVENTION

Electronic cigarette is an analog cigarette, by using heating wire to heat and vaporize fragrance-containing materials to produce aerosol for users smoking Referring to FIG. 1, a conventional electronic cigarette includes a connecting element 100 independently disposed. One end of the connecting element 100 is tensioningly assembled on the inhalation rod 200 and the other end of the connecting element 100 is threaded connected to a battery rod, thus making the inhalation rod 200 and the battery rod assembly together. However, the way of the connecting element 100 tensioningly assembled on the inhalation rod 200 affects the connecting firmness with the inhalation rod 200 and adversely affects the sealing of the e-liquid in the inhalation rod, therefore affects the normal use of the product. On the other hand, the incoherence between the connector 100 and the inhalation rod 200 and the surface of the battery rod adversely affects the appearance of the product and then results in inconvenience of use.

### SUMMARY OF THE INVENTION

A technical problem of the present invention to be solved is to provide an electronic cigarette inhalation rod and electronic cigarette with a stable structure, and convenient assembly

To solve the above-mentioned technical problem, the present invention provides an electronic cigarette inhalation rod includes an inhalation tube and a first connector disposed at one end of the inhalation tube. The inhalation tube and the first connector are an integrally formed structure. Therefore, by the integral formed structure of the inhalation tube and the first connector, on one hand the first connector will not fall down from the inhalation rod, the product is of stable structure, good quality, and easy to use; on the other hand, the assembly is convenient and the appearance is beautiful.

Furthermore, the inhalation tube disposes an atomization device. The atomization device includes a liquid-storage member and a guide pipe orderly nested and coaxially arranged, an atomization chamber formed in the guide pipe, an atomizer disposed in the atomization chamber, and an atomization base accommodating a distal end of the liquid-storage member and the guide pipe; An inhalation cover is disposed at the other end of the inhalation tube and accommodating another distal end of the liquid-storage member and the guide pipe. The center of the atomization base and the inhalation cover forms a venthole communicating with the atomization chamber.

Furthermore, the first connector is a threaded connector.

Furthermore, the first connector is a bayonet connector.

Furthermore, the first connector is a lock connector.

Furthermore, the first connector is a push-pull connector.

Furthermore, the first connector is an in-line connector.

On the surface of the first connector disposes male thread.

The first connector and the inhalation tube are cylinder structure, and the outer diameter of the first connector is smaller than the inter diameter of the inhalation tube. The first connector is a threaded column.

The atomizer comprises a liquid-guiding member with two ends respectively extending out the sidewall of the guide pipe and abutting to the liquid-storage member, and a heating wire winding on the outer surface of the liquid-guiding member; the first connector disposes inner electrode therein, the first connector serves as an outer electrode, and an insulation ring is disposed between the inner and outer electrodes.

The conductivity of the first connector serving as the outer electrode is achieved by using metal to make the first connector to obtain conductivity; or by coating conductive coatings on the inner surface or the outer surface of the first connector to obtain conductivity; or by disposing conductive wire or conductive strip on the inner surface or the outer surface to obtain conductivity thereof.

In addition, the present invention also provides an electronic cigarette, including a battery rod, and the aforementioned electronic cigarette inhalation rod. The electronic cigarette inhalation rod and the battery rod are matchingly connected.

Furthermore, the battery rod comprises a battery cylinder and a battery disposed in the battery cylinder. One end of the battery cylinder disposes a second connector which is matchingly connected with the first connector. Specifically, the connection structure of the second connector and the first connector matches each other.

Furthermore, the second connector serves as an outer electrode of the battery rod and disposes an inner electrode of the battery rod. An insulation ring is disposed between the outer electrode and the inner electrode of the battery rod.

Furthermore, the second connector disposes internal thread to match with the external thread of the first connector. The second connector and the battery cylinder are integrally formed.

Furthermore, the other end of the battery cylinder disposes a switch module and a light cap assembly.

The advantages of the embodiment of the present invention are: by adopting the integrally forming structure of the inhalation tube and the first connector, therefore achieves technical effects of stable structure product and convenient assembly.

### BRIEF DESCRIPTION OF THE DRAWINGS

FIG. 1 is a schematic view of assembly structure of an inhalation rod and an external thread of a conventional electronic cigarette.

FIG. 2 is a schematic view of the whole structure of an electronic cigarette according to an embodiment of the present invention.

FIG. 3 is a schematic view of an inhalation rod of an electronic cigarette according to the embodiment of the present invention.

FIG. 4 is a sectional view along line A-A of the inhalation rod of the electronic cigarette shown in FIG. 3.

FIG. 5 is an exploded, isometric view of the electronic cigarette shown in FIG. 2.

FIG. 6 is a sectional view along line B-B of the electronic cigarette shown in FIG. 2.

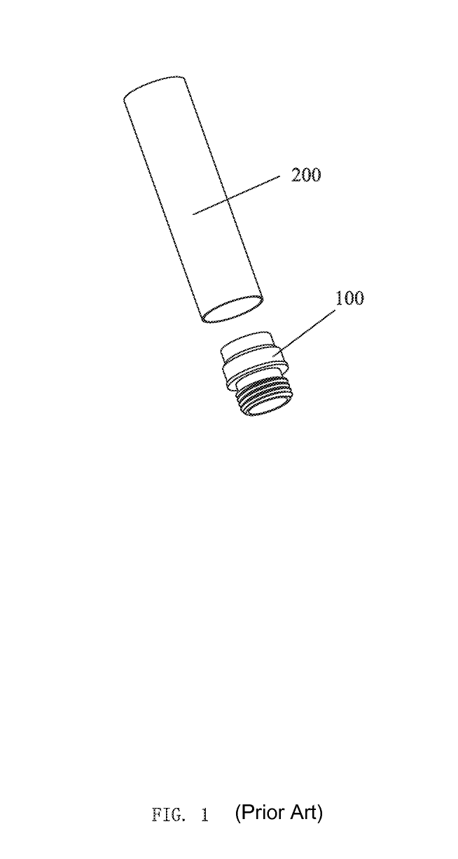

### DETAILED DESCRIPTION OF THE INVENTION

It is needed to state that embodiments and all element limitations in all embodiments may be combined in the case of no conflicts. The present invention will be described in detail in the following combining figures and embodiments.

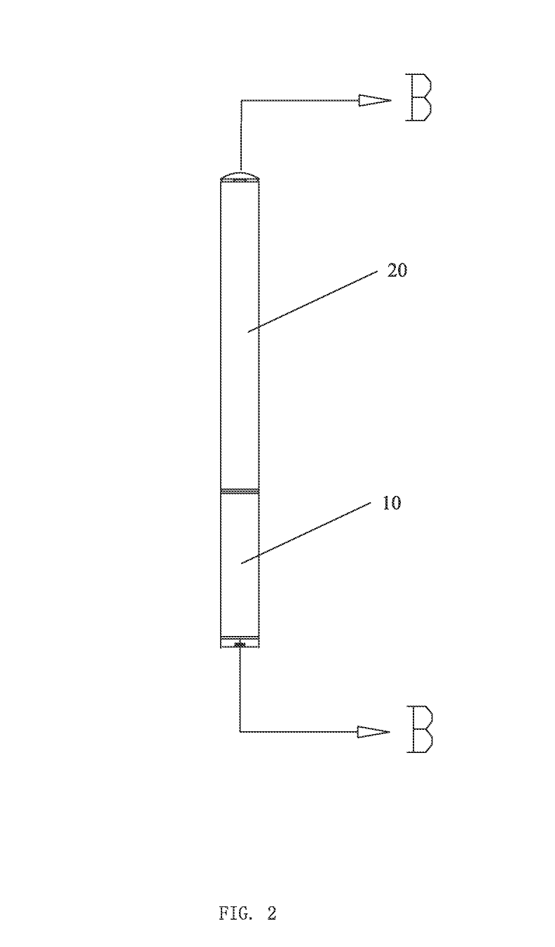

Referring to FIG. 2, the present invention provides an electronic cigarette inhalation rod 10 and an electronic cigarette, wherein the electronic cigarette includes an inhalation rod 10 and a battery rod 20 which are mutual butted.

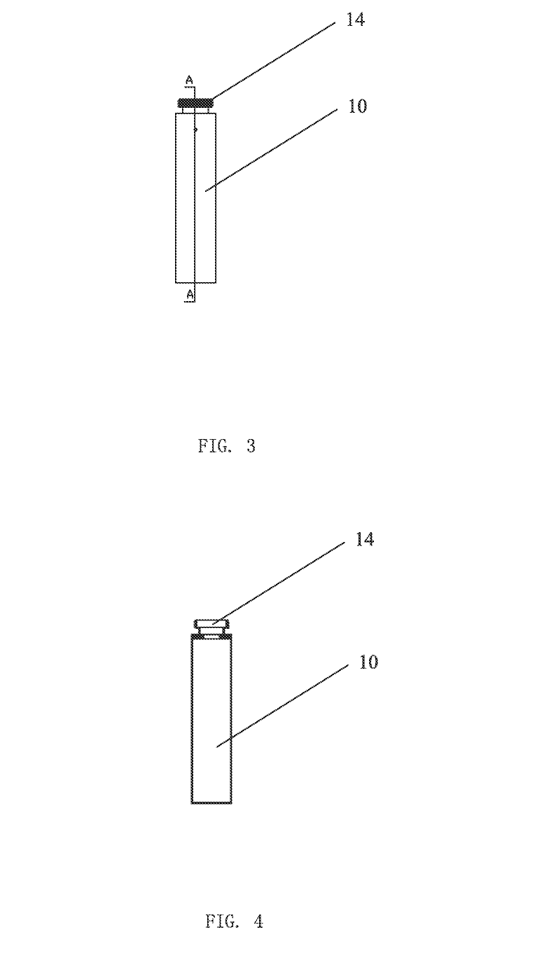

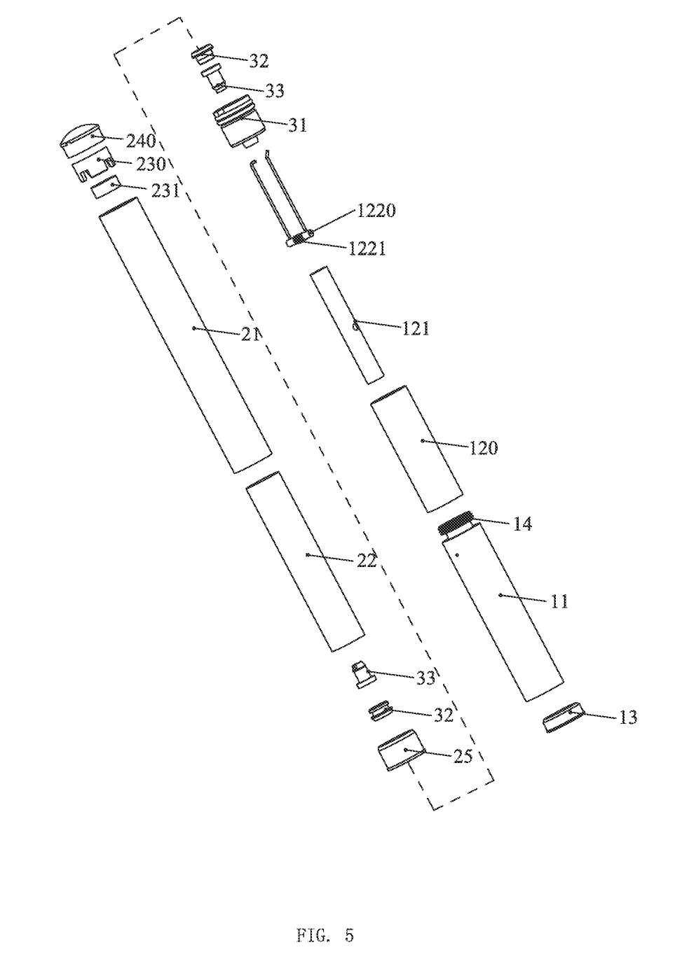

Referring to FIG. 3 to FIG. 6, the inhalation rod 10 includes a hollow cylindrical inhalation tube 11, an atomization device 12 arranged in the inhalation tube 11 and used to produce aerosol, an inhalation cover 13 arranged at one end (as an inhalation end) of the inhalation tube 11, and a first connector 14 arranged at the other end (as a connecting end) of the inhalation tube 11 and used to connect with the battery rod 20. As an embodiment, a sticker is sheathed on the outer surface of the inhalation tube (not shown).

In the embodiment, the inhalation tube 11 and the first connectors 14 is an integrally formed structure. Therefore, by the integrally formed structure between the inhalation tube 11 and the first connector 14, on one hand the product is of stable structure, good quality, and easy to use; on the other hand, no bulge happens, and the assembly is convenient and the appearance is beautiful. The first connector 14 is a threaded connector, which in essence is a hollow threaded column formed by the connecting end of the inhalation tube 11 coaxially extending along the length direction. The outer diameter of the first connector 14 is smaller than the outer diameter of the inhalation tube 11 such that the electronic cigarette surface formed by the abutting between the inhalation rod 10 and the battery rod 20 is a flat surface after the first connector 14 is plugged into the connecting end of the battery rod 20, therefore no connection portion protrudes from the outer surface of the electronic cigarette.

It is understood that the first connector 14 can also be any kind of bayonet connector, lock connector, push-pull connector and in-line connector.

The atomization device 12 includes a liquid-storage member 120, a guide pipe 121, an atomizer 122 and an atomization base 31. The liquid-storage member 120 and the guide pipe 121 are orderly and coaxially nested in the inhalation tube 11. The guide pipe 121 is supported in the inner wall of the liquid-storage member 120. Preferably the guide pipe 121 and the liquid-storage member 120 are tensioningly coordinated, and two ends of the guide pipe 121 and the liquid-storage member 120 are fixed by the atomization base 122 and the inhalation cover 13 respectively. The liquid-storage member 120 with e-liquid is sealed between the inner wall of the inhalation tube 11 and the outer wall of the guide pipe 121, therefore forming airflow channel in the center to guide and communicate aerosol and ambient air. The hollow cavity of the guide pipe 121 forms an atomization chamber 123, and the atomizer 122 is disposed in the atomization chamber 123. The inhalation cover 13 and the atomization base 31 can be tensioningly assembled in the inner wall of the inhalation tube 11. The inhalation cover 13 covers on the inhalation end of the inhalation tube 11, and defines air vent to communicate with the atomization chamber 123 to export aerosol. One distal end of the liquid-storage member 120 and the guide pipe 121 abuts on the inhalation cover 13. The atomization base 31 is substantially a cylinder, and defines a center venthole to communicate with the atomization chamber 123. The atomization base 31 includes a body 310. One end of the body 310 facing the atomization chamber 123 disposes an annular groove 314 to accommodate another distal end of the liquid-storage member 120 and the guide pipe 121. The other end of the body 310 disposes a through groove 312 along a radial direction communicating with the center venthole of the atomization base 31 to guide airflow. The atomization base 31 tensioningly assembled in the inner wall of the inhalation tube 11 and located adjacent to the connecting end of the inhalation tube 11. The liquid-storage member 120 and another distal end of the guide pipe 121 abut to the atomization base 31.

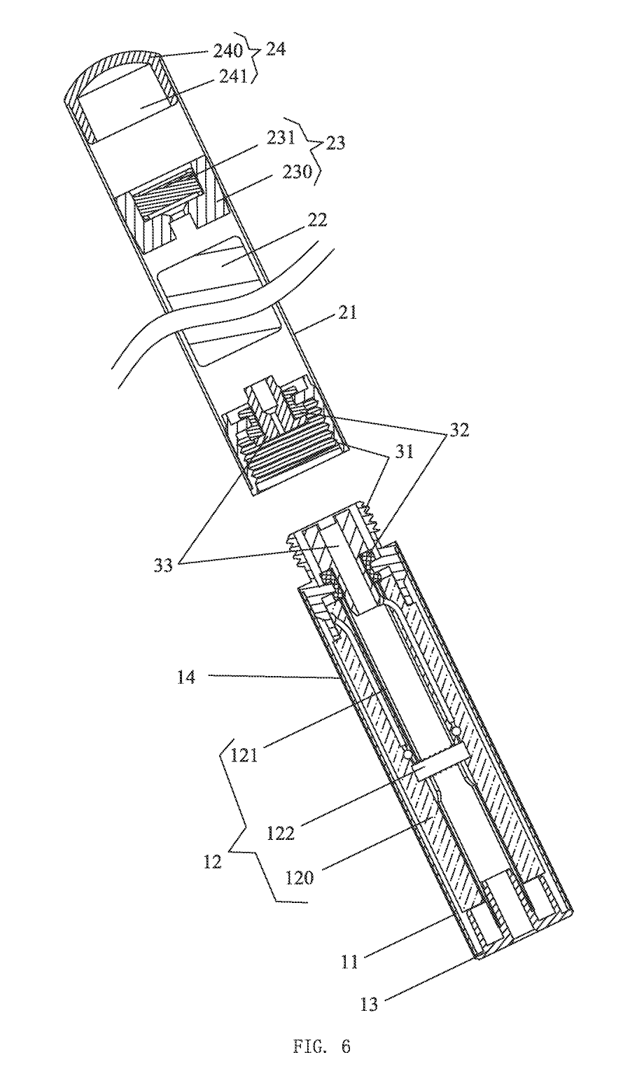

The liquid-storage member 121 is used to absorb and storage e-liquid, and is an annual column. Referring to FIG. 6, in order to seal e-liquid and avoid air-leakage for the better, a sealing ring 16 is further disposed between the inhalation cover 13 and the distal end of the guide pipe 121 and the liquid-storage member 120.

The atomizer 122 includes a liquid-guiding member 1220, of which two ends extend out of the outer sidewall of the guide pipe 121 and abut with the liquid-storage member 120, and a heating wire 1221 winding on the outer surface of the liquid-guiding member 1220.

In the embodiment, the first connector 14 also serves as an outer electrode of the atomizer 122, and an inner electrode 33 is sheathed in the first connector 14. An insulation ring 32 is padded between the outer electrode and the inner electrode 33. The two ends of the heating wire 1221 are electrically connected with the inner electrode 33 and the first connector 14 respectively. In the embodiment, the liquid-storage member 120 is a storage cotton, and the guide pipe 121 is a fiberglass tube; and the liquid-guiding device 1220 is a guiding wick.

In the present invention, conductivity of the first connector 14 serving as the outer electrode may be achieved by using conductive materials such as metal to make the first connector 14, and also may be achieved by coating conductive coatings on the inner surface or the outer surface thereof to achieve conductivity; and also may dispose separate conductive elements such as disposing conductive strip or conductive wire on the inner surface or the outer surface of the first connector 14.

The battery rod 20 includes a battery cylinder 21 and a battery 22 disposed in the battery cylinder 21. One end of the battery cylinder 21 disposes a second connector 25 mating and connecting with the first connector 14, and the other end of the battery cylinder 21 disposes a switch module 23 and a light cap assembly 24. The switch module 23 includes a support 230 and an airflow sensor 231 fixed on the support 230. The light cap assembly 24 includes a light cap 240 and an LED light 241 disposed inside of the light cap 240, and being red and used to simulate burning cigarette for state indication disposed.

The second connector 25 is fixed in the inner wall of the connecting end of the battery rod 20 by tensioningly assembled or other fixed methods. The second connector 25 is a cylinder with internal thread disposed inside thereof. The internal thread matches with the external thread of the first connector 14 of the inhalation rod 10. When the inhalation rod 10 is assembled on the battery rod 20, the inhalation rod 10 and the battery rod 20 are connected to be an integral structure by the external thread of the first connector 14 mating, matching, or engaging with internal thread of the second connector 25, and the connecting surface of the inhalation rod 10 and the battery rod 20 is smooth. In the embodiment, the second connector 25 serves as the outer electrode of the battery 22, and is sheathed with an inner electrode 33 therein and padded with an insulation ring 32therein. When the inhalation rod 10 and the battery rod 20 are assembled together, the first connector 14 and the second connector 25 serve as outer electrodes are simultaneously electrically connected, and the two inner electrodes 33 mutual abut, therefore achieves the electrical connection between the atomizer 122 and the battery 22. The center of the two inner electrodes 33 disposes venthole to communicate with the air path in the inhalation rod 10.

It is understood that the second connector 25 also may be integrally formed with the battery cylinder 21, or forming a connecting mechanism in the connecting end of the battery cylinder 21 to match with the connecting mechanism of the first connector 14 to serve as the second connector, such as internal thread, bayonet mechanisms and so on.

Embodiments of the present invention is shown and described in the above-mentioned. Various improvement and modifications can be made to the embodiments by those skilled in the art without departing from the true spirit and scope of the disclosure. The scope of the present invention is defined by the appended claims and equivalents thereof.

What is claimed is:

1. An electronic cigarette inhalation rod comprising:
an inhalation tube;
a first connector integrally formed with the inhalation tube and located at one end of the inhalation tube;
an atomization device disposed in the inhalation tube, the atomization device including a liquid-storage member and a guide pipe orderly nested and coaxially arranged, an atomization chamber formed in the guide pipe, an atomizer disposed in the atomization chamber, and an atomization base accommodating a distal end of the liquid-storage member and the guide pipe; and
an inhalation cover disposed at the other end of the inhalation tube and accommodating another distal end of the liquid-storage member and the guide pipe;
wherein the center of the atomization base and the inhalation cover defines a venthole communicating with the atomization chamber.

2. According to the electronic cigarette inhalation rod in claim 1, wherein the first connector is a threaded connector, a bayonet connector, a lock connector, a push-pull connector, or an in-line connector.

3. According to the electronic cigarette inhalation rod in claim 1, wherein the surface of the first connector disposes external thread.

4. According to the electronic cigarette inhalation rod in claim 3, wherein the first connector and the inhalation tube are cylinder structures, and an outer diameter of the first connector is smaller than the outer diameter of the inhalation tube; the first connector is a threaded column.

5. According to the electronic cigarette inhalation rod in claim 1, wherein the atomizer comprises a liquid-guiding member with two ends respectively extending out the sidewall of the guide pipe and abutting to the liquid-storage member, and a heating wire winding on the outer surface of the liquid-guiding member; the first connector disposes an inner electrode therein, the first connector serves as an outer electrode, and an insulation ring is disposed between the inner and outer electrodes.

6. According to the electronic cigarette inhalation rod in claim 5, wherein conductivity of the first connector serving as the outer electrode is achieved by using metal to make the first connector; or by coating conductive coatings on the inner surface or the outer surface of the first connector to obtain conductivity; or by disposing conductive wire or conductive strip on the inner surface or the outer surface to obtain conductivity.

7. An electronic cigarette comprising:
a battery rod; and
an electronic cigarette inhalation rod, the electronic cigarette inhalation rod comprising:
an inhalation tube;
a first connector integrally formed with the inhalation tube and located at one end of the inhalation tube;
an atomization device disposed in the inhalation tube, the atomization device including a liquid-storage member and a guide pipe orderly nested and coaxially arranged, an atomization chamber formed in the guide pipe, an atomizer disposed in the atomization chamber, and an atomization base accommodating a distal end of the liquid-storage member and the guide pipe; and
an inhalation cover disposed at the other end of the inhalation tube and accommodating another distal end of the liquid-storage member and the guide pipe;
wherein the center of the atomization base and the inhalation cover defines a venthole communicating with the atomization chamber; the electronic cigarette inhalation rod and the battery rod are matchingly connected.

8. According to the electronic cigarette in claim 7, wherein the battery rod comprises a battery cylinder and a battery disposed in the battery cylinder; one end of the battery cylinder disposes a second connector which is matchingly connected with the first connector.

9. According to the electronic cigarette in claim 8, wherein the second connector serves as an outer electrode of the battery rod, and disposes an inner electrode of the battery rod; an insulation ring is disposed between the outer electrode and the inner electrode of the battery rod.

10. According to the electronic cigarette in claim 7, wherein the first connector is a threaded connector, a bayonet connector, a lock connector, a push-pull connector, or an in-line connector.

11. According to the electronic cigarette in claim 7, wherein the surface of the first connector disposes external thread.

12. According to the electronic cigarette in claim 11, wherein the second connector disposes internal thread to match with the external thread of the first connector; the second connector and the battery cylinder are integral formed; the other end of the battery cylinder disposes a switch module and a light cap assembly.

13. According to the electronic cigarette in claim 11, wherein the first connector and the inhalation tube are cylinder structures, and an outer diameter of the first connector is smaller than the outer diameter of the inhalation tube; the first connector is a threaded column.

14. According to the electronic cigarette in claim 7, wherein the atomizer comprises a liquid-guiding member with two ends respectively extending out the sidewall of the guide pipe and abutting to the liquid-storage member, and a heating wire winding on the outer surface of the liquid-guiding member; the first connector disposes inner electrode therein, the first connector serves as an outer electrode, and an insulation ring is disposed between the inner and outer electrodes.

15. According to the electronic cigarette in claim 14, wherein conductivity of the first connector serving as the outer electrode is achieved by using metal to make the first connector; or by coating conductive coatings on the inner surface or the outer surface of the first connector to obtain conductivity; or by disposing conductive wire or conductive strip on the inner surface or the outer surface to obtain conductivity.

16. According to the electronic cigarette in claim 7, wherein the second connector serves as an outer electrode of the battery rod and disposes an inner electrode of the battery rod, and an insulation ring is disposed between the outer electrode and the inner electrode of the battery rod.

17. According to the electronic cigarette in claim 8, wherein the second connector is integrally formed with the battery cylinder.

18. According to the electronic cigarette in claim 11, wherein the switch module includes a support and an airflow sensor fixed on the support., and the light cap assembly includes a light cap and an LED light disposed inside of the light cap.

19. An electronic cigarette inhalation rod, comprising:
an inhalation tube;
a first connector integrally formed with the inhalation tube and located at one end of the inhalation tube;
an atomization device disposed in the inhalation tube; and
an inhalation cover disposed at the other end of the inhalation tube;
wherein the center of the inhalation cover defines a venthole communicating with the atomization device.

20. According to the electronic cigarette inhalation rod in claim 19, wherein the atomization device includes a liquid-storage member and a guide pipe orderly nested and coaxially arranged, an atomization chamber formed in the guide pipe, an atomizer disposed in the atomization chamber, and an atomization base accommodating a distal end of the liquid-storage member and the guide pipe; the inhalation cover accommodates another distal end of the liquid-storage member and the guide pipe.
